(19) United States
(12) Patent Application Publication
(10) Pub. No.: US 2010/0113902 A1
(43) Pub. Date: May 6, 2010

(54) Small Bore Magnetic Resonance Imaging Photoplethysmographic Sensor

(75) Inventors: Bernard F. Hete, Kittanning, PA (US); Eric W. Starr, Allison Park, PA (US); Chris Zawada, Oakmont, PA (US)

Correspondence Address: BLYNN L. SHIDELER;THE BLK LAW GROUP, 3500 BROKKTREE ROAD, SUITE 200, WEXFORD, PA, 15090, US

(73) Assignee: STARR LIFE SCIENCES CORP., Oakmont, PA (US)

(21) Appl. No.: 12/606,180

(22) Filed: Oct. 26, 2009

Related U.S. Application Data
(60) Provisional application No. 61/108,486, filed on Oct. 24, 2008.

(51) Int. Cl. A61B 5/1455; A61B 5/055

(52) U.S. Cl. 600/323; 600/411

(57) ABSTRACT

An efficient, effective, MRI compatible small bore MRI noninvasive photoplethysmographic sensor for animals such as small rodents, namely rats and mice. The photoplethysmographic sensor for animals comprising: a non-magnetic sensor coupling attachable to an animal; fiber optic cable coupled to the sensor coupling and configured to deliver a signal to and receive a signal from the animal tissue adjacent the sensor coupling; an opto-electical converter coupled to the fiber optic cable, the converter including a receiver coupled the fiber optic cable portion configured to receive a signal from the animal tissue and including an emitter coupled to the fiber optic portion configured to deliver a signal to the animal tissue; an electronic coupling extending from the opto-electric converter and configured to be coupled to the emitter and the receiver, wherein the electronic coupling is configured to extend outside of the MRI chamber; and a processor coupled to the electronic coupling.

The present invention claims priority of U.S. Provisional Patent Application Ser. No. 61/108,486 entitled “Small Bore Magnetic Resonance Imaging Photoplethysmographic Sensor” filed Oct. 24, 2008.

### BACKGROUND OF THE INVENTION

1. Field of the Invention

The present invention relates to photoplethysmographic readings for animal research and more particularly, the present invention is directed to a noninvasive photoplethysmographic sensor for small bore MRI applications such as small bore animal MRI research.

2. Background Information

Small Animal Photoplethysmography

A photoplethysmograph is an optically obtained plethysmograph, which, generically, is a measurement of changes in volume within an organ whole body, usually resulting from fluctuations in the amount of blood or air that the organ contains. A photoplethysmograph is often obtained by using a pulse oximeter.

A conventional pulse oximeter monitors the perfusion of blood to the dermis and subcutaneous tissue of the skin. Pulse oximetry is a non invasive method that allows for the monitoring of the oxygenation of a subject's arterial blood, generally a human or animal patient or an animal (or possibly human) research subject. The patient/research distinction is particularly important in animals where the data gathering is the primary focus, as opposed to care-giving, and where the physiologic data being obtained may, necessarily, be at extreme boundaries for the animal.

As a brief history of pulse oximetry, it has been reported that in 1935 an inventor Matthes developed the first 2-wavelength earlobe O₂ saturation meter with red and green filters, later switched to red and infrared filters. This was the first device to measure O₂ saturation. Further, in 1949, an inventor Wood added a pressure capsule to squeeze blood out of the earlobe to obtain zero setting in an effort to obtain absolute O₂ saturation value when blood was readmitted. The concept is similar to today's conventional pulse oximetry but suffered due to unstable photocells and light sources and the method was not used clinically. In 1964 an inventor Shaw assembled the first absolute reading ear oximeter by using eight wavelengths of light which was commercialized by Hewlett Packard. This use was limited to pulmonary functions due to cost and size.

Effectively, modern pulse oximetry was developed in 1972, by Aoyagi at Nihon Kohden using the ratio of red to infrared light absorption of pulsating components at the measuring site, and this design was essentially commercialized by BIOX/Ohmeda in 1981 and Nellcor, Inc. in 1983. Prior to the introduction of these commercial pulse oximeters, a patient's oxygenation was determined by a painful arterial blood gas, a single point measure which typically took a minimum of 20-30 minutes processing by a laboratory. It is worthy to note that in the absence of oxygenation, damage to the human brain starts in 5 minutes with brain death in a human beginning in another 10-15 minutes. Prior to its introduction, studies in anesthesia journals estimated US patient mortality as a consequence of undetected hypoxemia at 2,000 to 10,000 deaths per year, with no known estimate of patient morbidity. With essentially real time oxygenation results, pulse oximetry has become a standard of care for human patients since about 1987.

Pulse oximetry has also been a critical research tool for obtaining associated physiologic parameters in humans and animals beginning soon after rapid pulse oximetry became practical.

In conventional pulse oximetry, a sensor is placed on a thin part of the subject's anatomy, such as a human fingertip or earlobe, or in the case of a neonate, across a foot, and two wavelengths of light, generally red and infrared wavelengths, are passed from one side to the other. Changing absorbance of each of the two wavelengths is measured, allowing determination of the absorbance due to the pulsing artery alone, excluding venous blood, skin, bone, muscle, fat, etc. Based upon the ratio of changing absorbance of the red and infrared light caused by the difference in color between oxygen-bound (bright red) and oxygen unbound (dark red or blue, in severe cases) blood hemoglobin, a measure of oxygenation (the percent of hemoglobin molecules bound with oxygen molecules) can be made. The measured signals are also utilized to determine other physical parameters of the subjects, such as heart rate (pulse rate). Starr Life Sciences, Inc. has utilized pulse oximetry measurements to calculate other physiologic parameters such as breath rate, pulse distension, and breath distention, which can be particularly useful in various clinical and research applications.

In addressing animal pulse oximetry, particularly for small rodents, one approach has been to modify existing human or neonate oximeters for use with rodents. This approach has proven impractical as the human based systems can only stretch so far and this approach has limited the use of such adapted oximeters. For example, these adapted human oximeters for animals have an upper limit of heart range of around 400 or 450 beats per minute which is insufficient to address mice that have a conventional heart rate of 400-800 beats per minute. Starr Life Sciences has developed a small mammal oximeter, rather than an adapted human model, that has effective heart rate measurements up to 900 beats per minute and beyond, and this is commercially available under the Mouse Ox® oximeter brand.

Regarding animal pulse oximetry, consideration must be made for the particular subject or range of subjects in the design of the pulse oximeter, for example the sensor must fit the desired subject (e.g., a medical pulse oximeter for an adult human finger simply will not adequately fit onto a mouse). Consequently there can be significant design considerations in developing a pulse oximeter for small mammals or for neonates or for adult humans. Starr Life Sciences has developed pulse oximetry clips that are effective for use with animals such as small mice and rats, and these clips are applicable for the tails, neck, thighs, head of the animal (with the different intended clip application areas of the animal resulting in distinct advantages/disadvantages for the signal, and optionally some changes in the clip construction).

Small Bore Magnetic Resonance Imaging (MRI)

Magnetic resonance imaging (MRI), or Nuclear magnetic resonance imaging (NMRI), is primarily a medical imaging technique most commonly used in radiology to visualize the structure and function of the subject tissue. The MRI provides detailed images of the tissue in any plane. MRI provides much greater contrast between the different soft tissues of a body than computed tomography (CT) does, making it especially useful in neurological (brain), musculoskeletal, cardiovascular, and oncological (cancer) imaging. Unlike CT, MRI uses no ionizing radiation, but uses a powerful magnetic field to align the nuclear magnetization of, generally, hydrogen atoms in water in the tissue. Radiofrequency fields are used to systematically alter the alignment of this magnetization, causing the hydrogen nuclei to produce a rotating magnetic field detectable by the scanner. This signal can be manipulated by additional magnetic fields to build up enough information to construct an image of the body. Functional Magnetic Resonance Imaging (fMRI) is a type of specialized MRI scan and since the early 1990s, fMRI has come to dominate the brain mapping field.

MRI is a relatively new technology, which has been in use for little more than 30 years (at the time of filing this application, as compared with over 110 years for X-Ray radiography. The first MR Image was published in 1973 and the first study performed on a human took place in 1977. Magnetic resonance imaging was developed from knowledge gained in the study of nuclear magnetic resonance. In its early years the technique was referred to as nuclear magnetic resonance imaging (NMRI). However, as the word nuclear was associated in the public mind with radiation exposure, thus it is generally now referred to simply as MRI.

Some research groups have used clinical MR scanners for imaging small animal models. However, it has been found to be difficult to achieve a reasonable spatial resolution at an acceptable signal-to-noise ratio with these “large bore” scanners. Specialized “small-bore” animal MRI scanners are available for high-resolution MRI of small animals. In the drug development fields a discussion of the development and application of small bore MRIs for imaging of small animals can be found in the Markus Rudin 2005 book entitled “Imaging in Drug Discovery and Early Clinical Trials” available at http://www.springer.com.

Bruker Biospin has a line of small bore Animal MRI Solutions for Molecular and Preclinical Imaging—see www.bruker-biospin.com. Additionally, horizontal small bore preclinical MRI systems are available, for example, from M2M Imaging Corporation (See www.m2mimaging.com.

The studies conducted in small bore MRIs have proven very beneficial. For a representative example the Science Daily, on Oct. 2, 2008, announced that a new magnetic resonance imaging procedure performed on a small bore MRI can detect very early breast cancer in mice, including ductal carcinoma in situ (DCIS), a precursor to invasive cancer. Some of the tumors detected were less than 300 microns in diameter, the smallest cancers ever detected by MRI and this was using a small bore MRI. The initial study was done with a small-bore MRI system with a 4.7 Tesla magnet, about twice the strength of a high-end clinical imaging device. The research team has begun using a new 9.4 Tesla small bore MRI.

In light of these possibilities a number of research facilities have small bore MRI systems for research. The University of British Columbia MRI Research center provides a small bore MRI facility which is located in its Life Sciences Centre. The facility aims to help academic and industrial scientists meet their research goals through the use of MRI technology, particularly for the study of animal models of human disease and disorder. The facility is equipped with a 7 Tesla Bruker-Biospec small bore MRI scanner with a 30 cm wide cylindrical bore. The compatible specimen size is dependent on the size of the gradient coils chosen for the experiment. For typical applications gradients coils of 12 cm or 20 cm inner diameter are used.

The Athinoula A. Martinos Center for Biomedical Imaging is located on the Massachusetts General Hospital Research Campus in the Charlestown Navy Yard (CNY) and includes a collection of small bore MRI laboratories (see www.nmr.mgh.harvard.edu/martinos).

The MRI facility at the Howard Florey Institute in Autralia has a small bore MRI that it makes available to outside researchers. The 4.7 Tesla Magnetic Resonance Imaging (MRI) scanner was installed in September 1999 and is available for small animal scanning. The Howard Florey Institute (HFI) (also called Australia's brain research institute) welcomes enquiries from scientists who wish to access the Facility on a service basis, as well as researchers wishing to collaborate with its specialists. (See www.florey.edu.au).

Case Western Reserve University has the Case Center for Imaging Research and accommodates the imaging of small animals with small bore MRIs, such as the Bruker Biospec® Animal MRI Systems which includes Two Bruker Biospec® MRI systems, 7 T and 9.4 T. These high-field systems are equipped with multiple gradient sets specifically for in-vivo micro-imaging applications (<100 um). The Biospec® systems are also equipped with broadband capabilities for multinuclear imaging and spectroscopy applications (ex. 1H, 31P, 15N, 13C, 19F). These systems will also be capable of cardiac/respiratory gating/monitoring to limit the negative effects of motion on image quality. This feature is described as critical for imaging animals with high respiration/heart rates. (see http://ccir.uhrad.com/sairc/mri.asp).

The Neuroimaging Research Group at the 7 T MRI/MRS facility opened in June 2003 in the Garscube Estate of the University of Glasgow and provides a small bore MRI facility. The facility serves as a national resource for Scotland. The facility contacts leading researchers who may have an interest in using the facility. The described major advantages of 7 T small bore MRI, as asserted by this group, is that it is a non-invasive imaging technique with resolutions down to 40 microns or less and which permits serial studies to be performed in the living animal. The system can produce images based on 30 or more physico-chemical parameters which are used for examining tissue morphology, blood flow, metabolism and chemistry in vivo. Applications include drug evaluation, transgenic phenotyping and stem cell tracking. The primary use of the facility will be in biomedical research applications involving the in vivo imaging of small animals but the high image resolutions and multiplicity of parameters measurable by the system also have applications in plant, materials and food science and the study of some industrial process dynamics.

The above cites examples are merely representative of some of the small bore MRI facilities currently in use. Numerous other hospitals, universities and research institutions provide small bore MRI facilities.

MRI Tools

In all MRI systems, tools and other equipment within the MRI room must be MRI compatible. MRI compatible can be broken down into two components. The first is equipment that is MRI safe, and this term means that the tool will not be effected by or, more importantly damage the MRI equipment. For example, because of the extremely powerful magnets in these environments, generally only non-ferrous materials can be near the MRI bore. A ferrous containing element, such as a wrist watch or metal scalpel, if, mistakenly taken into the MRI room can be ripped from the users wrist or out of the users hand by the magnet and accelerated until it crashes into the MRI housing, possibly damaging the MRI housing. Furthermore, the MRI magnets would need to be ramped down in a time consuming and costly procedure so that the metal object could be removed from the housing. Thus, ferrous containing materials are generally considered not MRI safe.

A second part of MRI compatibility is equipment that does not affect the imaging of the MRI equipment. Electrical cables within the MRI bore can detrimentally affect the image as electricity can induce an additional magnetic field. Consequently, electrical cables need to be kept form the MRI bore.

There remains a need for efficient, effective sensors which adequately address laboratory animal research applications using animals in small bore MRI environments and more particularly, there is a need for efficient, effective, MRI compatible small bore MRI noninvasive photoplethysmographic sensor for animals such as small rodents, namely rats and mice.

It is an object of the present invention to address the deficiencies of the prior art discussed above and to do so in an efficient, cost effective manner.

### SUMMARY OF THE INVENTION

The various embodiments and examples of the present invention as presented herein are understood to be illustrative of the present invention and not restrictive thereof and are non-limiting with respect to the scope of the invention.

According to one non-limiting embodiment of the present invention, an efficient, effective, MRI compatible small bore MRI noninvasive photoplethysmographic sensor for animals such as small rodents, namely rats and mice. In one non-limiting aspect of the present invention the fiber optic line includes turning of the light pipe 90 degrees at the animal attaching member to allow less bulk at animal contact point. In one non-limiting aspect of the present invention the system places an opto-electronic coupler between the wire and the optical fiber with the ratio of wire to fiber being variable. In one non-limiting aspect of the present invention the system uses standard interface connecters on the opto-electronic coupler enclosure to allow variable length wire and/or fiber optics. In one non-limiting aspect of the present invention the system uses coupling at the box to allow the user to have different length electrical wire and/or fiber-optics.

These and other advantages of the present invention will be clarified in the description of the preferred embodiments taken together with the attached figures.

### BRIEF DESCRIPTION OF THE DRAWINGS

FIG. 1 is a schematic representation of the small bore MRI photoplethysmographic sensor for animals such as small rodents, namely rats and mice, in accordance with one embodiment of the present invention;

FIG. 2 is an enlarged schematic representation of the opto-electronic coupler for use in the system of FIG. 1;

FIG. 3. is a view of the Fiber Bundle with Bent Ends for use in the system of FIG. 1;

FIG. 4 is a view of prism technology for a further modification of the present invention;

FIG. 5 is one alternative embodiment for the animal attaching portion of the present invention;

FIG. 6 is one alternative embodiment for the animal attaching portion of the present invention;

FIG. 7 is one alternative embodiment for the animal attaching portion of the present invention; and

FIG. 8 is and end view of the embodiment of FIG. 7 of the present invention.

### DESCRIPTION OF THE PREFERRED EMBODIMENTS

In summary, the present invention relates to a small bore photoplethysmographic sensor system 10 for animals, such as rats and mice that are utilized in a laboratory environment. Photoplethysmographic measurements on laboratory animals in a small bore MRI 16 have been difficult which limits the research that can be conducted. Further, in the pulse oximetry field there has been a lack of adequate photoplethysmographic sensors for small mice (and even small rats), until the advent of the Mouse Ox® brand pulse oximeters by Starr Life Sciences. Prior to this development, commercially available pulse oximeters could provide heart rate data up to about 350 or 450 beats per minute (and even this range required special software modifications for some sensors), which were basically suitable for rats but not small mice given that the small mouse will have heart rates in the range of 400 to 800 beats per minute. The Mouse Ox™ brand of pulse oximeters for small rodents has an effective range up to (currently) about 900-1000 beats per minute which has opened up a wider selection of subjects for this type of research.

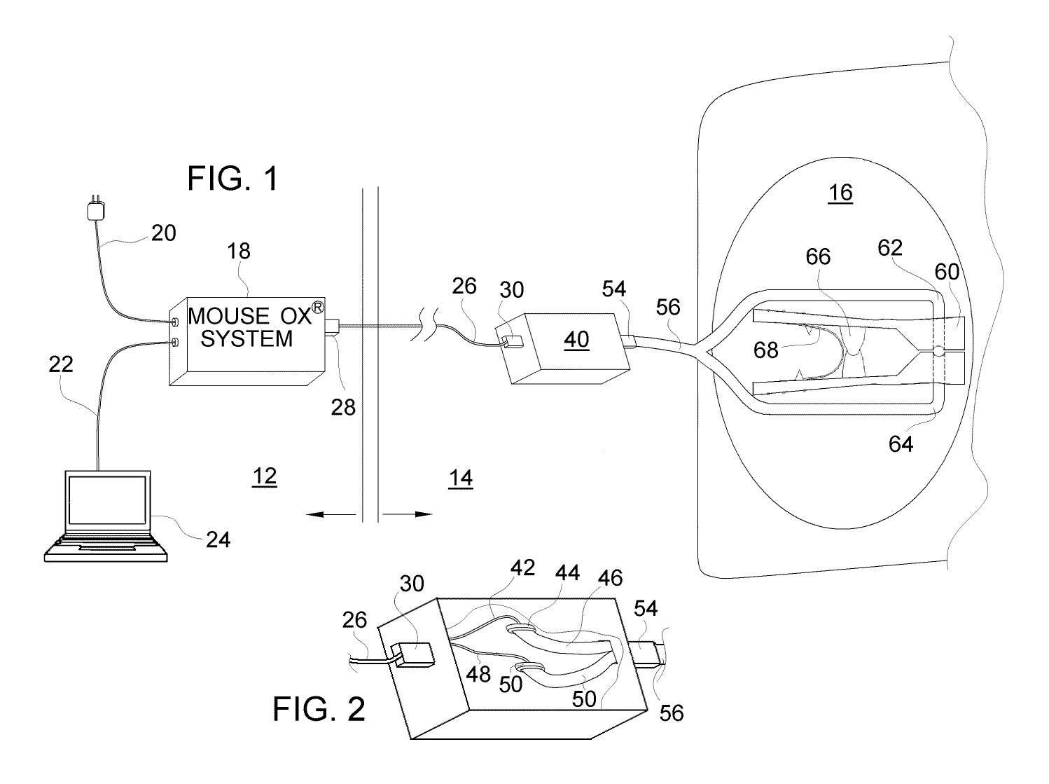

FIG. 1 is a schematic representation of a small bore MRI photoplethysmographic sensor 10 for animals such as small rodents, namely rats and mice, in accordance with one embodiment of the present invention. The system 10 is for use in a MRI chamber 14 with a portion in the test room 12, wherein the Small Bore MRI 16 is in the chamber 14.

The system 10 uses the Mouse Ox® system or processor 18 such as available in October 2008 from Starr Life Sciences. The system 10 may use a conventional power source such as through plug 20 or the system 10 could use a battery power source. The system 10 couples to a computer 24, such as a lap top, through coupling 22. The computer 24 will process, record, and display the results of the system 10 in a conventional fashion for animal pulse oximeters as known in the art.

A conventional electronic coupling 26 with standard plugs 28 and 30 extends from the processor 18 to an opto-electronic converter 40. As shown in FIG. 2 the converter 32 houses an LED emitter 44 connected to the coupling 26 through connection 42. The converter 32 houses an optical receiver 50 connected to the coupling 26 through connection 48.

Fiber optic couplings 46 and 52 extend from the emitter 44 and the receiver 50, respectively, and extend to the coupling or plug 54 of the fiber optic bundle 56. Coupling or plug 54 preferably allows the fiber optic bundle to be disconnected from the converter 40. In this manner the user can easily thread the bundle 56 through a small bore environment without the attached converter 40 which could hamper the set up procedure.

The fiber optic bundle 56 extends to a plastic clip 60 that may be biased via plastic spring 68 through a hinge 66. Each fiber optic bundle 56 extends through a 90 degree bend 62 and 64. The length of fiber optic bundle 56 is only long enough to reach into the small bore 16 preferably less than 10′, or more preferably less than 6′. However the converter 40 and disconnect plug 54 allow the bundle 56 to be of any desired length, and a variety of lengths can be supplied in accordance with user needs. In a similar manner the plugs 30 and 28 can allow for a variety of lengths of cables 26 to be used with the device to provide the distance from the processor 18 to the converter 40 that is desired.

The subject animal may be any subject animal for which photoplethysmographic measurements are desired. A large amount of laboratory research is conducted on rats and mice, however prior to the Mouse Ox® brand of sensor photoplethysmographic measurements have been of limited availability to the researchers when using such subjects. Consequently, the present invention has particular application to research associated with rats and mice. More accurately the present invention provides particular advantages and expands potential research possibilities when utilized with subjects of the order rodentia, and even more precisely, when utilized with the sub-order muroidia. A particularly advantageous aspect of the present invention is that the system 10 allows for photoplethysmographic measurements from an animal in a small bore MRI 16.

In this application the controller or processor 18 is the commercially available MouseOx® product from Starr Life Sciences with the unique sensor mounting and coupling as described hereinafter. The details of the controller 18, including the user interface, the user display on computer 24 is not discussed herein in detail.

STARR Life Sciences has recently introduced the Small Bore MRI Sensor system 10, which plugs directly into the MouseOx® controller 18 and functions without any special equipment or software, the system 10 shown in FIG. 1 described above. The Small Bore MRI Sensor system 10 can be used in any small bore MRI environment 16. The sensor itself is preferably about 20 feet in length so that the MouseOx® and computer can be located a safe distance from the MRI device 16. The end of the sensor contains a 5 foot non-magnetic portion formed by cable 56 to clip 60 that can be located inside any small bore MRI machine and can be used during imaging without affecting the quality of the images. The sensor clips to the animal via the non-magnetic plastic clip 60 in exactly the same manner as one would use a standard MouseOx® sensor clip.

Fiber Bundle with Bent Ends

One embodiment of an MRI sensor according to the present invention is to implement a section of optical fiber bundle that terminates at the animal end in a sharp 90° bend as shown in FIG. 1. The advantage of this configuration is that the light traveling down the fibers does not have to pass from the fibers through another entity or entities before it reaches the tissues. This is important because each transmission of light across a boundary with different indices of refraction (IOR) causes a loss of transmission based on the degree of difference in IOR between the two interfaces.

One alternative in this fiber bundle with bent ends is shown in FIG. 3 in which the bent ends are formed from a fiber optic material that has a very tight bending radius, such as a boro silica glass fiber. Boro silica glass allows for a tight bending radius applicable for this application, however it has a high signal attenuation over distance. In order to minimize the attenuation issue with boro silica fibers, shortly after the bent end the fiber can be coupled to a glass silica fiber having low signal attenuation over distance from the point right behind the bent ends to the coupling or plug 54.

Fiber Bundle with Prisms and Lenses

Prisms

The difficulty associated with bent ends of a fiber bundle is in making the right angle turn tight enough. In the small animal MRI application, there is little room for the fiber to make a right angle turn and the profile of a clip placed on the animal tissue 80 needs to be as small as possible. As noted above switching to a fiber that allows for a tight radius as boro silica can alleviate this issue.

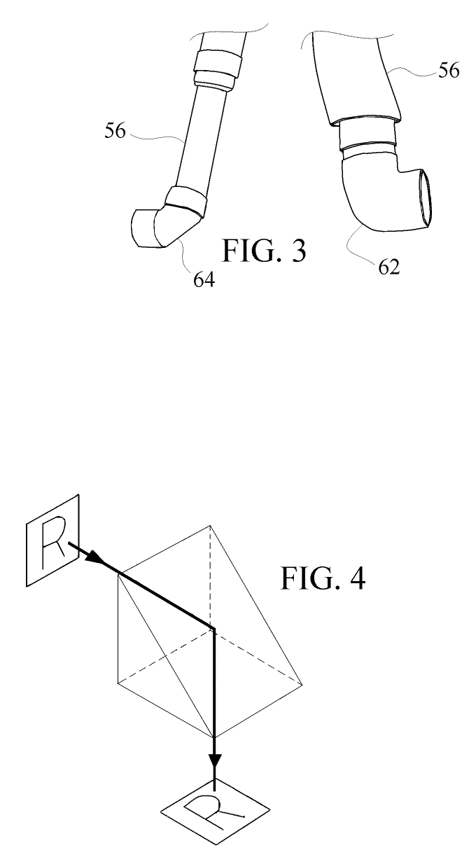

Another option is to use a 90° prism 70, as shown in FIGS. 4-8. In this type of lens, the hypotenuse of the triangle is a mirrored surface, allowing light that shines into one face to be reflected at a right angle out of the other face.

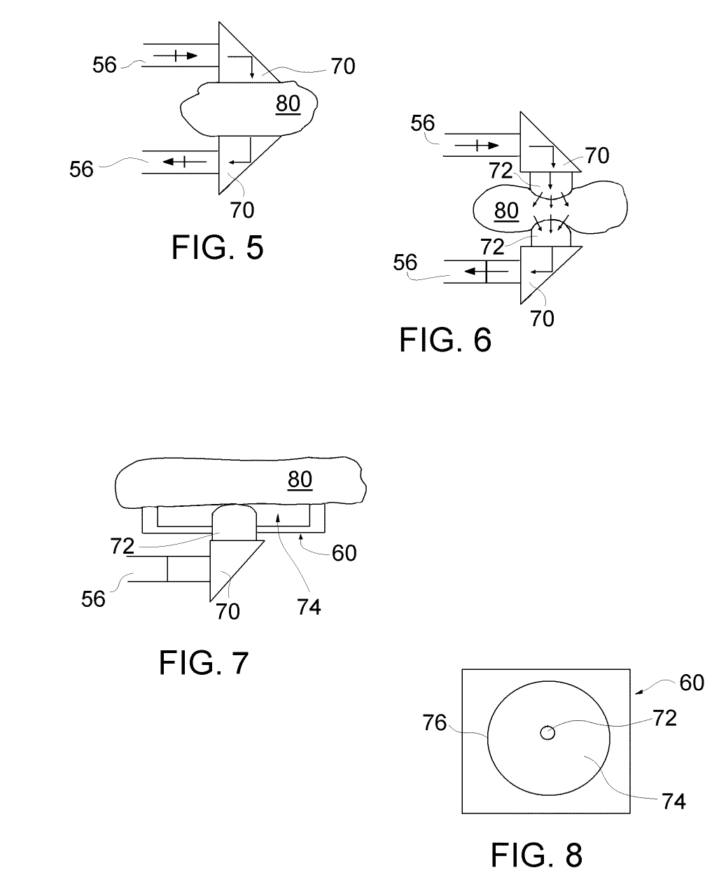

Thus, for transmission of light, the LED fiber 56 bundle end would be optically coupled to the prism 70, and the right angle face would point at the tissue 80 as shown in FIG. 5. On the receiver side, another prism 70 would be placed to collect light transmitted through the tissue 80, and the other optical fiber 56 would be coupled to the right angle face to pass to the photodiode. The principle is demonstrated in the FIGS. 4-5.

Lenses

To further improve the transmission and gathering of light, one can use what is called a drum lens 72 on the non-fiber face of the prism 72 as shown in. A drum lens 72 is a glass cylinder that is flat on one end, and has a hemispherical shape on the other. By using this lens shape, light can be better dispersed on the LED side and it can be better collected on the photodiode, or receiver, side. See rightward figure below.

Tissue Coupling Lens

The one difficulty with the drum lens 72 configuration shown in the figure above is that protrusion of the drum lenses is important in order to transmit and receive light from around the hemisphere, but protrusion into the tissue causes pressure points that can limit blood flow in that region.

To circumvent this problem, an alternative special lens has been designed that allows protrusion of the drum lens, but permits light to enter from the sides, while maintaining a flat face against the tissue. Diagrams of this appears in the figures.

In the embodiment that we have assembled, we spot face the holding clip half and provide a hole through the clip half through which the drum lens will protrude so that it is flush with the top of the clip. After we adhere the drum lens and prism to the clip, we fill the spot face with an optical coupling material that further holds the drum lens in place, but more importantly, allows light to enter from the sides while still maintaining a flat interface to contact the tissue. This flat interface eliminates points of pressure concentration.

There are many ways to implement a strategy like this, but the key is to have some shape such as the circular one shown (it could be square or other), that has a cross dimension that is larger than the diameter of the drum lens. The depth of the optical coupling pool needs to be a minimum of the radius of the drum lens hemisphere, while the maximum depth could be anything.

Lastly, the material that comprises the optical coupling material could be some sort of gel, an optically clear glue, or even a hard material. The only requirement is that it transmits light and provides a good IOR match with the drum lens. We use an optical UV-cured glue that has low viscosity. It is poured into the void and cured under a UV light.

Can use coupling at the box to allow the user to have different length electrical wire and/or fiber-optics.

A further modification of the invention is to use the bent ends of the fiber optic fibers 56 discussed in connection with FIGS. 1-3 with a lens or diffuser. One diffuser that has proven effective is to use a small translucent portion of the clip itself as a diffusing lens. Further in this embodiment, having the clip formed with a small pocket of material adjacent the tissue in front of the bent ends of the fiber optic fibers that holds diffusing or optical coupling gel has proved to be effective.

The above described invention provides an MRI compatible small bore MRI noninvasive photoplethysmographic sensor 10 for animals comprising: a non-magnetic sensor coupling, such as a clip 60, attachable to an animal tissue 80; a fiber optic cable 56 coupled to the coupling (clip 60) and configured to deliver a signal to the animal tissue 80 adjacent the coupling (clip 60) and to receive a signal from the animal tissue 18 adjacent the coupling (clip 60); an opto-electical converter 40 coupled to the fiber optic cable 56, the converter 40 including a receiver 50 coupled the fiber optic cable 56 portion configured to receive a signal from the animal tissue 18 and including an emitter 44 coupled to the fiber optic portion 56 configured to deliver a signal to the animal tissue 18; an electronic coupling 26 extending from the opto-electric converter 40 and configured to be coupled to the emitter 44 and the receiver 50, wherein the electronic coupling 26 is configured to extend outside of the MRI chamber 14; and a processor 18 coupled to the electronic coupling 26.

The MRI compatible small bore MRI noninvasive photoplethysmographic sensor according to the invention can be described as wherein the animal coupling is a clip 60 that is configured to have two clip faces on opposed sides the animal tissue 18 as shown with one fiber optic portion 56 configured to deliver a signal to the animal tissue on one clip face and one fiber optic portion 56 configured to receive a signal from the animal tissue 18 on an opposed clip face forming a transmissive system.

The clip 60 may a plastic clip to easily form a non-magnetic material attaching mechanism. An adhesive strip type coupling could also be used for the sensor coupling.

The MRI compatible small bore MRI noninvasive photoplethysmographic sensor according to the invention may provide that the one fiber optic portion configured to deliver a signal to the animal tissue on one clip face and one fiber optic portion configured to receive a signal from the animal tissue on an opposed clip face are each bent approximately 90 degrees in the area of the clip face. The fiber optic portion adjacent the clip faces may be formed of boro silica material in the area adjacent the clip.

The MRI compatible small bore MRI noninvasive photoplethysmographic sensor according to the invention may further including a diffuser between the fiber optic material and the animal tissue. The MRI compatible small bore MRI noninvasive photoplethysmographic sensor according to invention may also provide that the fiber optic cable is coupled to the converter through a disconnect plug.

The MRI compatible small bore MRI noninvasive photoplethysmographic sensor according to invention may provide that the fiber optic cable has a length of less than 10 feet, or even less than 6 feet.

Whereas particular embodiments of the invention have been described above for purposes of illustration, it will be evident to those skilled in the art that numerous variations of the details of the present invention may be made without departing from the spirit and scope of the present invention.

What is claimed is:

1. A photoplethysmographic sensor for animals for use in a small bore MRI.

2. An MRI compatible small bore MRI noninvasive photoplethysmographic sensor for animals comprising:
a non-magnetic sensor coupling attachable to an animal;
fiber optic cable coupled to the sensor coupling and configured to deliver a signal to the animal tissue adjacent the sensor coupling and to receive a signal from the animal tissue adjacent the sensor coupling;
an opto-electical converter coupled to the fiber optic cable, the converter including a receiver coupled the fiber optic cable portion configured to receive a signal from the animal tissue and including an emitter coupled to the fiber optic portion configured to deliver a signal to the animal tissue;
an electronic coupling extending from the opto-electric converter and configured to be coupled to the emitter and the receiver, wherein the electronic coupling is configured to extend outside of the MRI chamber;
a processor coupled to the electronic coupling.

3. The MRI compatible small bore MRI noninvasive photoplethysmographic sensor according to claim 2 wherein the animal coupling is a clip that is configured to have two clip faces on opposed sides the animal tissue with one fiber optic portion configured to deliver a signal to the animal tissue on one clip face and one fiber optic portion configured to receive a signal from the animal tissue on an opposed clip face.

4. The MRI compatible small bore MRI noninvasive photoplethysmographic sensor according to claim 3 wherein the clip is a plastic clip.

5. The MRI compatible small bore MRI noninvasive photoplethysmographic sensor according to claim 3 wherein the one fiber optic portion configured to deliver a signal to the animal tissue on one clip face and one fiber optic portion configured to receive a signal from the animal tissue on an opposed clip face are each bent approximately 90 degrees in the area of the clip face.

6. The MRI compatible small bore MRI noninvasive photoplethysmographic sensor according to claim 5 wherein the fiber optic portion is formed of boro silica material in the area adjacent the clip.

7. The MRI compatible small bore MRI noninvasive photoplethysmographic sensor according to claim 5 further including a diffuser between the fiber optic material and the animal tissue.

8. The MRI compatible small bore MRI noninvasive photoplethysmographic sensor according to claim 5 wherein the fiber optic cable is coupled to the converter through a disconnect plug.

9. The MRI compatible small bore MRI noninvasive photoplethysmographic sensor according to claim 5 wherein the fiber optic cable has a length of less than 10 feet.

10. The MRI compatible small bore MRI noninvasive photoplethysmographic sensor according to claim 5 wherein the fiber optic cable has a length of less than 6 feet.

11. The MRI compatible small bore MRI noninvasive photoplethysmographic sensor according to claim 10 wherein the processor is configured to calculate heart rates up to 900 beats per minute.

12. The MRI compatible small bore MRI noninvasive photoplethysmographic sensor according to claim 11 wherein the processor is coupled to a lap top computer.

13. The MRI compatible small bore MRI noninvasive photoplethysmographic sensor according to claim 3 wherein variable lengths of fiber optic cable can be implemented in the sensor.

14. The MRI compatible small bore MRI noninvasive photoplethysmographic sensor according to claim 3 wherein variable lengths of electrical connector can be implemented in the sensor.

15. The MRI compatible small bore MRI noninvasive photoplethysmographic sensor according to claim 3 further including a prism between the fiber optic material and the animal tissue.

16. The MRI compatible small bore MRI noninvasive photoplethysmographic sensor according to claim 3 further including a lens between the end of the fiber optic material and the animal tissue.

17. An MRI compatible small bore MRI noninvasive photoplethysmographic sensor for animals comprising:
a plastic clip attachable to an animal, wherein the clip is configured to have two clip faces on opposed sides the animal tissue;
fiber optic cable coupled to the clip, wherein with one fiber optic portion is configured to deliver a signal to the animal tissue on one clip face and one fiber optic portion configured to receive a signal from the animal tissue on an opposed clip face;
an opto-electical converter coupled to the fiber optic cable, the converter including a receiver coupled the fiber optic cable portion configured to receive a signal from the animal tissue and including an emitter coupled to the fiber optic portion configured to deliver a signal to the animal tissue;
an electronic coupling extending from the opto-electric converter and configured to be coupled to the emitter and the receiver, wherein the electronic coupling is configured to extend outside of the MRI chamber; and
a processor coupled to the electronic coupling, wherein the processor is configured to calculate heart rates above beats per minute.

18. The MRI compatible small bore MRI noninvasive photoplethysmographic sensor according to claim 17 wherein variable lengths of fiber optic cable can be implemented in the sensor.

19. The MRI compatible small bore MRI noninvasive photoplethysmographic sensor according to claim 17 wherein variable lengths of electrical connector can be implemented in the sensor.

20. The MRI compatible small bore MRI noninvasive photoplethysmographic sensor according to claim 17 wherein the one fiber optic portion configured to deliver a signal to the animal tissue on one clip face and one fiber optic portion configured to receive a signal from the animal tissue on an opposed clip face are each bent approximately 90 degrees in the area of the clip face.
